(19) United States
(12) Patent Application Publication
(10) Pub. No.: US 2015/0190454 A1
(43) Pub. Date: Jul. 9, 2015

(54) Herbal Composition and a Method for the Treatment of Qi Stagnancy and Blood Stasis Type of Hyperlipidemia

(71) Applicant: Zhenbo Fan, Chiping (CN)

(72) Inventor: Zhenbo Fan, Chiping (CN)

(21) Appl. No.: 14/585,527

(22) Filed: Dec. 30, 2014

(30) Foreign Application Priority Data
Jan. 5, 2014 (CN) 201410002847.3

(51) Int. Cl. A61K 36/9066; A61K 36/752; A61K 36/79; A61K 36/75; A61K 35/64; A61K 36/48; A61K 36/00; A61K 36/738; A61K 36/44; A61K 36/77; A61K 36/575; A61K 36/8905; A61K 36/537; A61K 36/704; A61K 36/533; A61K 36/37; A61K 36/236; A61K 36/21; A61K 36/36; A61K 36/286; A61K 36/45; A61K 36/31; A61K 36/483; A61K 36/328; A61K 36/486; A61K 36/324; A61K 36/53; A61K 36/47; A61K 36/736; A61K 36/484; A61K 36/185

(52) U.S. Cl. CPC A61K 36/9066; A61K 36/185; A61K 36/752; A61K 36/79; A61K 36/75; A61K 35/64; A61K 36/48; A61K 36/00; A61K 36/738; A61K 36/44; A61K 36/77; A61K 36/575; A61K 36/8905; A61K 36/537; A61K 36/704; A61K 36/533; A61K 36/37; A61K 36/236; A61K 36/21; A61K 36/36; A61K 36/286; A61K 36/45; A61K 36/31; A61K 36/483; A61K 36/328; A61K 36/486; A61K 36/324; A61K 36/53; A61K 36/47; A61K 36/736; A61K 36/484

(57) ABSTRACT

A composition and a method for treating Qi stagnancy and blood stasis type of hyperlipidemia. The composition comprises in various parts: akebia fruit, curcuma root, pomelo, fructus aurantii, fructus aurantii immaturus, star anise, murraya paniculata, aspongopus, sword beans, caesalpinia decapetala, China fir, finger citron, rose, green tangerine peel, sundew, trifoliate orange, kaki calyx, litchi, litchi nucleus, magnolia, cyperus rotundus, citron fruit, Chinese buckeye seed, salvia multiorrhiza, polygonum orientale, motherowrt fruit, euonymus alatus, campsis grandiflora, rhizome of Sichuan lovage, medical cyathula root, vaccaria, Chinese rose flower, balsamine, safflower, azaleas, semen brassicae campestris, caesalpinia sappan, gleditsia spine, myrrh, caulis spatholobi, frankincense, herba lycopi, selaginella, tanarius major, peach seed, and licorice. The method comprises weighing predetermined amount of the aforementioned herbs, blending the herbs into 1800 ml of water and soak for 30 minutes, boiling the mixture with gentle heat for 30 minutes, and filtering the residue to achieve 390 ml of liquid.

The present application hereby claims priority to Chinese application No. 201410002847.3, filed on Jan. 5, 2014.

### TECHNICAL FIELD

The present invention relates to Chinese medicine compositions, and a method for medical preparation, and more specifically speaking, it is a preparation, and a method for the treatment of Qi stagnancy and blood stasis type of hyperlipidemia.

### BACKGROUND OF THE INVENTION

Blood stasis is an important pathology of many disease processes in Traditional Chinese Medicine (“TCM”). Simply put, it means that the flow of blood is slowed down and brought to a static state. Under TCM, blood is believed to be stored in the liver, and propelled by the heart Qi to flow through the body. If blood circulation is stagnant or slowed down by certain factors, it will lead to retention of blood in any part of the body or overflow of blood out of the vessels, resulting in blood stasis. Blood stasis frequently occurs in long-term chronic illnesses, and due to various etiologies it may be the root of many age-related disharmonies. It can also be commonly observed after surgery or external traumatic injuries.

According to TCM, Qi stagnation is the most common cause of blood stasis.

Blood and Qi are like Yin and Yang. Qi provides energy to move blood through the vessels and holds the Blood within them. Conversely, blood is essential in the formation of Qi and provides transportation for Qi into all areas of the body. Any significant disturbance of these actions can cause the impedance of Qi flow. Blood will then stagnate and cease flowing, and as a result, blood stasis is formed. Common causes of Qi stagnation include emotional causes, injuries, exogenous pathogenic factors, phlegm, Qi deficiency, and blood deficiency.

The name of “hyperlipidemia” does not exist in TCM, but it has long been recognized and referred as “phlegm,” “fat,” “stroke,” “vertigo,” and other diseases under the “dirty blood disease” category. According to TCM, “dirty” blood is loaded with toxins or waste products, which is caused by turbid Qi, phlegm dampness and stasis in blood. It is recorded in Confucian's Duties To their Parents that, “people who eat fine food such as fatty meats and refined grains, will be harmed by the food and drinks they consumed, and may develop flatulence, fullness and heartburn.” TCM believes that one of the causes of this disease is patients' low spirit and depression of Qi for a long term, obstructed channels and vessels, disorders in lipid transfusion, dysfunction of excretion, as well as blood stasis and Qi stagnancy.

Nowadays, lipid regulating drugs that are generally used for the treatment of Qi stagnancy and blood stasis type of hyperlipidemia are listed as follows:

1. Bezafibrate: The most common adverse reactions were gastrointestinal discomfort, such as indigestion, anorexia, nausea, vomiting, and a sense of fullness. It shall not be used in patients who are allergic to the drug, or in patients who suffer from gallbladder diseases, cholelithiasis, hepatic insufficiency or primary biliary cirrhosis, severe renal insufficiency, and hypoalbuminemia caused by nephrotic syndrome. It shall be used with caution in pregnant and lactating women.

2. Acipimox: Initial treatment can cause skin vasodilation, increase sensitivity to heat, such as hot flashes over the face or itching all over the body which would usually disappear in a few days after treatment, without stopping the medication. It shall not be used in patients who are allergic to this drug, or who have gastrointestinal ulcers. Pregnant women, lactating women, or children are forbidden to use this drug. Before using this medicine, patients should have low-cholesterol and low-fat diets and quit drinking Dosage shall be reduced in patients with renal insufficiency.
3. Alginic Sodium Diester: The probability of occurrence of adverse reactions is 5%-23%, which mainly are palpitations, angina pectoris, hypotensions and electrocardiography (ECG) abnormalities. Possible adverse reactions also include xerostomia, nausea, vomiting, diarrhea, abdominal pain, constipation and loss of appetite. In some cases, patients may also develop liver abnormalities manifested as elevation in thymol turbidity test (TTT) or serum alanine aminotransferase test (ALT), skin redness, itching, rash, annular erythema and erythroderma, extremity venous varix, limb peripheral edema, acute laryngeal edema and anaphylactic shock. It shall not be used in patients with bleeding disorder or bleeding tendency, severe liver and kidney dysfunction. It shall be used with caution in patients with hypotension, hypovolemia, thrombocytopenia, non-hyperviscosity syndrome, non-platelet aggregation hyperactivity, and allergic constitution.
4. Fenofibrate: The probability of occurrence of adverse reactions is about 2%-15%, among which gastrointestinal reactions and insomnia are the common reactions. Contraindications include patients with known allergic reactions to this drug, a history of gallbladder disease, cholelithiasis, severe renal insufficiency, hepatic dysfunction, primary biliary cirrhosis, or unexplained chronic liver function abnormalities. It shall be used with caution in patients with gallstone, liver dysfunction and renal insufficiency. Patients need to stop using the product if serum amylase, or blood cholesterol, low-density lipoprotein level elevates while using. This product is not a substitute for diet therapy, and diet control is still necessary. If the product has to be used in combination with hydroxy methyl coenzyme A (HMG-CoA) reductase inhibitors, the patients shall be closely monitored for serum creatine kinase (CK) levels. In case of significantly increased CK values or suspected myopathy or rhabdomyolysis, the usage of the product shall be immediately stopped. Patients shall be informed to consult a doctor immediately when they experience muscle pain, tenderness, or muscle weakness because sometimes they lead to very serious consequences.
5. Fluvastatin: Adverse reaction is a mild and transient indigestion. Patients who suffer active hepatitis or unexplained persistent elevation in serum transaminase, pregnant and lactating women, as well as patients under 18 years old are forbidden to use this product. Alcoholics, patients with past history of liver diseases, and those who are allergic to other HMG-CoA reductase inhibitors are suggested to use the product with caution. When this product is used for treatment, diet therapy must be carried out simultaneously. In case of persistent elevation in serum transaminases, and more than three times over the upper limit, or significantly increased creatine kinase or myositis, this drug shall be discontinued immediately.
6. Gemfibrozil: The most common side effect is gastrointestinal discomfort, such as indigestion, anorexia, nausea, vomiting, fullness, and stomach discomfort. Patients with known allergic reactions to this drug, or patients who suffer from gallbladder disease, cholelithiasis, liver dysfunction or primary biliary cirrhosis, severe renal insufficiency, and reduction of serum proteins caused by nephrotic syndrome are forbidden to use the product. The product also interferes with the diagnosis, which may lead to a reduction of hemoglobin, hematocrit, or white blood cells, or an elevation of blood creatine phosphokinase, alkaline phosphatase, aminotransferase, or lactate dehydrogenase. After discontinuing the use of the product, the blood cholesterol and triglyceride levels may rebound over the original levels, so that a low-fat diet shall be provided and blood lipids shall be monitored until they reach normal levels.
7. Lovastatin: The most common side effect is gastrointestinal discomfort, such as, diarrhea and flatulence, and also includes headaches, rashes, dizziness, blurred vision and taste disorders. It can cause reversible elevated serum aminotransferase. Hence it is necessary to monitor patients' liver functions. Patients with known allergic reactions to this product, or patients who have active hepatitis or unexplained persistent elevation in serum aminotransferase are forbidden to use the product. Patients who are allergic to other HMG-CoA reductase inhibitors should use this product with caution. In case of an elevation of serum aminotransferase more than three times over the upper limit while using the product, or a significant increase of creatine kinase or myositis and pancreatitis, patients shall discontinue the usage immediately. When using this product, if patients experience hypotension, severe acute infection, trauma or metabolic disorders, it shall be noted that renal failure may occur secondary to myolysis.
8. Pravastatin: Adverse reactions include mildly elevated aminotransferase, rash, myalgia, headache, chest pain, nausea, vomiting, diarrhea, fatigue and so on. Patients with known allergic reactions to this drug, or patients who have active hepatitis or persistent elevation in liver function tests, as well as pregnant and lactating women are forbidden to use the drug. Patients with a history of liver disease or drinking history, as well as patients who are allergic to other HMG-CoA reductase inhibitors shall use the product with caution. It has poor efficacy for treating patients who have homozygous familial hypercholesterolemia. During treatment, liver function should be checked regularly. If persistent ALT or aspartate transaminase (AST) elevation equals or exceeds three times over the upper limit, treatment shall be discontinued. Use of HMG-CoA reductase inhibitor type of lipid-lowering drugs may occasionally cause elevated creatine phosphokinase (CPK). If the elevated value of CPK is ten times over three times of the normal upper limit, the usage of the product shall be discontinued. In case of unexplained muscle pain, tenderness, and weakness, especially accompanied by malaise and fever, attention should be drawn. In case of hypotension, severe acute infection, trauma and metabolic disorders when using this product, it shall be noted that renal failure may occur secondary to myolysis.
9. Simvastatin is generally well tolerated with mild and transient adverse reactions in most patients. It is prohibited to be used by patients who are allergic to any component, pregnant women, or patients with active hepatitis or unexplained persistent elevation in serum aminotransferase. It shall be used with caution if used in combination with tetrahydronaphthol type of calcium channel blocker Mibefradil. It shall be used with caution in patients who consume excessive alcohol or have a previous history of liver diseases. The conditions of diffused muscle pain, muscle weakness and/or significant CK elevation (more than ten times greater than normal value) should be considered to be myopathy, and the treatment shall be immediately discontinued. For patients with acute or serious conditions suggestive of myopathy and a tendency of acute renal failure caused by rhabdomyolysis, HMG-CoA reductase inhibitor treatment shall be discontinued. Due to complete lack of low-density lipoprotein (LDL) receptors in patients with homozygous familial hypercholesterolemia, the effect of simvastatin treatment is not ideal for such patients.
10. Vitamin E nicotinate: Adverse reactions may include warm feelings on the neck and facial area, skin redness, headaches and other reactions. Patients may also experience serious skin flushing, itching and gastrointestinal discomfort. Patients with arterial bleeding, diabetes, glaucoma, gout, hyperuricemia, liver diseases, ulcer diseases, and hypotension need to use the product with caution.

The adverse effects of the above treatments, and the usage limitations in certain group of patients are overcome by the present invention.

### SUMMARY OF THE INVENTION

To overcome the aforementioned shortcomings, the present invention is to provide a preparation, and a method for the treatment of Qi stagnancy and blood stasis type of hyperlipidemia. Here, hyperlipidemia refers to lipid metabolism disorders, abnormal fat metabolism or transportation, and too high lipid concentration in plasma, including total cholesterol and triglyceride levels higher than those of normal people of the same age. The clinical conditions of Qi stagnancy and blood stasis type of hyperlipidemia is emotional depression, irritability, chest tightness and more sighing, chest or breast fullness, channeling pain, abdominal pain, impetuous temper, or irregular menstruation, dysmenorrhea, or difficulty in swallowing and spitting, dark purple tongue, or ecchymosis, and unsmooth pulse.

Unlike Western medicines, TCM formulas comprise many components, usually extracts of raw herbs with each one present in very small quantity. The belief is that there is synergism among different components. One advantage of this kind of formulation is to avoid excessive toxicity when any one component is given in large quantity. The present invention is prepared by forty-six kinds of herbs that are common and can be purchased in any TCM pharmacy. The finished liquid medicine can be easily made and has small side effects with a shorter course of treatment and a higher cure rate. These herbs assist and guide each other, and could avoid the adverse effects, allergic reactions and toxicity caused by antibiotics in Western medicine.

To obtain the forgoing object, the technical solution is provided as follows.

A Chinese medicine preparation, and a method for the treatment of Qi stagnancy and blood stasis type of hyperlipidemia most preferably comprising by weight: 5 parts of akebia fruit, 6 parts of curcuma root, 6 parts of pomelo, 6 parts of fructus aurantii, 6 parts of fructus aurantii immaturus, 6 parts of star anise, 6 parts of murraya paniculata, 6 parts of aspongopus, 6 parts of sword beans, 6 parts of caesalpinia decapetala, 6 parts of China fir, 6 parts of finger citron, 5 parts of rose, 6 parts of green tangerine peel, 5 parts of sundew, 5 parts of trifoliate orange, 6 parts of kaki calyx, 6 parts of litchi, 6 parts of litchi nucleus, 6 parts of magnolia, 6 parts of cyperus rotundus, 2 parts of citron fruit, 6 parts of Chinese buckeye seed, 2 parts of salvia multiorrhiza, 6 parts of polygonum orientale, 3 parts of motherowrt fruit, 2 parts of euonymus alatus, 6 parts of campsis grandiflora, 6 parts of rhizome of Sichuan lovage, 6 parts of medical cyathula root, 6 parts of vaccaria, 6 parts of Chinese rose flower, 5 parts of balsamine, 6 parts of safflower, 5 parts of azaleas, 1 parts of semen brassicae campestris, 6 parts of caesalpinia sappan, 5 parts of gleditsia spine, 6 parts of myrrh, 5 parts of caulis spatholobi, 6 parts of frankincense, 6 parts of herba lycopi, 6 parts of selaginella, 5 parts of tanarius major, 6 parts of peach seed, and 9 parts of licorice.

The processed method of the preparation of TCM for the treatment of Qi stagnancy and blood stasis type of hyperlipidemia comprises the steps as follows: (a) Providing the raw materials as follows: 5-10 parts of akebia fruit, 6-12 parts of curcuma root, 6-12 parts of pomelo, 6-12 parts of fructus aurantii, 6-12 parts of fructus aurantii immaturus, 6-12 parts of star anise, 6-12 parts of murraya paniculata, 6-12 parts of aspongopus, 6-12 parts of sword beans, 6-12 parts of caesalpinia decapetala, 6-12 parts of China fir, 6-12 parts of finger citron, 5-10 parts of rose, 6-12 parts of green tangerine peel, 5-10 parts of sundew, 5-10 parts of trifoliate orange, 6-12 parts of kaki calyx, 6-12 parts of litchi, 6-12 parts of litchi nucleus, 6-12 parts of magnolia, 6-12 parts of cyperus rotundus, 2-4 parts of citron fruit, 6-12 parts of Chinese buckeye seed, 2-4 parts of salvia multiorrhiza, 6-12 parts of polygonum orientale, 3-6 parts of motherowrt fruit, 2-4 parts of euonymus alatus, 6-12 parts of campsis grandiflora, 6-12 parts of rhizome of Sichuan lovage, 6-12 parts of medical cyathula root, 6-12 parts of vaccaria, 6-12 parts of Chinese rose flower, 5-10 parts of balsamine, 6-12 parts of safflower, 5-10 parts of azaleas, 1-2 parts of semen brassicae campestris, 6-12 parts of caesalpinia sappan, 5-10 parts of gleditsia spine, 6-12 parts of myrrh, 5-10 parts of caulis spatholobi, 6-12 parts of frankincense, 6-12 parts of herba lycopi, 6-12 parts of selaginella, 5-10 parts of tanarius major, 6-12 parts of peach seed, and 9-18 parts of licorice; (b) blend the provided raw materials into 1800 ml of water, and soak for 30 minutes; (c) boil the medicinal herbs with gentle heat for 30 minutes; (d) filter the residue to achieve 390 ml of liquid, which is the Chinese medicine for treatment of Qi stagnancy and blood stasis type hyperlipidemia. When a patient needs treatment for Qi stagnancy and blood stasis type of hyperlipidemia, the drug liquid shall be adjusted to 35° C. He or she needs to consume 130 ml of liquid medicine each time, three times a day, and two days for one treatment course. If symptoms still exist, a second course of treatment is required until curing.

Among the forty-six kinds of herbs, akebia fruit, curcuma root, pomelo, fructus aurantii, fructus aurantii immaturus, star anise, murraya paniculata, aspongopus, sword beans, caesalpinia decapetala, China fir, finger citron, rose, green tangerine peel, sundew, trifoliate orange, kaki calyx, litchi, litchi nucleus, magnolia, cyperus rotundus, citron fruit, Chinese buckeye seed, have effects of inducing diuresis for removing edema, speeding up the metabolism and clearing toxic materials, relieving fidgets, and reducing pain. These twenty-three herbs play a predominant role in treating Qi stagnancy and blood stasis type of hyperlipidemia, so they are principal drugs.

Salvia multiorrhiza, polygonum orientale, motherwort fruit, euonymus alatus, campsis grandiflora, rhizome of Sichuan lovage, medical cyathula root, vaccaria, Chinese rose flower, balsamine, safflower, azaleas, semen brassicae campestris, caesalpinia sappan, gleditsia spine, myrrh, caulis spatholobi, frankincense, herba lycopi, selaginella, and tanarius major, have the effects of replenishing vital essence, regulating the stomach and alleviating pain, reducing sweat, dissipating dampness and easing heat stroke, invigorating spleen and normalizing the function of stomach, helping to produce saliva and slake thirst, as well as pain relieving. These twenty-one herbs could assist the principal drugs in treatment of Qi stagnancy and blood stasis type of hyperlipidemia, so they are ministerial drugs.

Traditionally, peach seed is believed to be able to lead various drugs directly to lesions, so it is used as an adjuvant drug. Licorice could relieve spasm and pain, clear away toxic materials, and coordinate the nature of drugs. It is believed to be able to reduce the toxicity of various drugs, so it is used as a guiding drug.

These forty-six herbs are applied with compatibility, together achieving the effects of inducing diuresis for removing edema, clearing away heat and toxic materials, dampness jaundice, inducing diuresis for treating stranguria, clearing heat and relieving fidgets, reducing fever and inducing diruesis, removing dampness and promoting diuresis, nourishing the marrow and essence, strengthening the spleen and nourishing the stomach, clearing liver and improving vision, isolation clear and discharging turbid, nourishing blood to tranquilize mind, easing the potency of drugs, expelling pus and dispersing swelling, invigorating spleen and normalizing the function of stomach as well as pain relieving. With proper compatibility in these herbs, this drug is the best formula for the treatment of Qi stagnancy and blood stasis type of hyperlipidemia.

### EXAMPLES

There are 369 patients with Qi stagnancy and blood stasis type of hyperlipidemia in total, 226 males, accounting for 61.3%, and 143 females, accounting for 38.7%.

TABLE 1
Age range for 369 patients with Qi stagnancy and
blood stasis type of hyperlipidemia

Age

| | 30~35 | 36~40 | 41~45 | 46~50 |
|---|---|---|---|---|
| Number of cases | 193 | 49 | 121 | 6 |
| Percentage | 52.3% | 13.3% | 32.8% | 1.6% |

TABLE 2
Efficacy in 369 patients with Qi stagnancy and
blood stasis type of hyperlipidemia

| | Efficacy | | |
|---|---|---|---|
| | Cured | Improved | Invalid |
| Number of cases | 366 | 3 | No |
| Percentage | 99.2% | 0.8% | No |

TABLE 3
Treatment course for 369 patients with Qi stagnancy and
blood stasis type of hyperlipidemia

Treatment days

| | 3~5 | 6~9 | 10~14 | 15~20 |
|---|---|---|---|---|
| Number of cases | 213 | 29 | 122 | 5 |
| Percentage | 57.7% | 7.9% | 33.1% | 1.3% |

TABLE 4
Before and after treatment in 369 patients with Qi stagnancy and blood stasis type hyperlipidemia

| | | | After treatment | | |
|---|---|---|---|---|---|
| | | Before treatment | Cured | Improved | Invalid |
| Clinical conditions | | Emotional depression, irritability, chest | No emotional | Occasional emotional | Same with the |
| | | tightness and more sighing, chest or breast | depression or irritability, | depression and irritability, | conditions before |
| | | fullness, channeling pain, abdominal pain, | no abdominal pain, no | occasional abdominal pain, | treatment |
| | | impetuous temper, or irregular menstruation, | dysmenorrhea, normal | occasional dysmenorrhea, | |
| | | dysmenorrhea, or difficulty in swallowing and | tongue, thin and white | slightly dark purple tongue, | |
| | | spitting, dark purple tongue, or ecchymosis, | tongue coating, stringy | slightly unsmooth pulse. | |
| | | and unsmooth pulse. | pulse. | | |
| Blood test | Total | Increase | Normal | Slight increase | Same with the |
| | cholesterol | | | | conditions before |
| | | | | | treatment |
| | Triglyceride | Increase | Normal | Slight increase | Same with the |
| | | | | | conditions before |
| | | | | | treatment |

What is claimed is:

1. A composition for treating Qi stagnancy and blood stasis type hyperlipidemia, wherein the hyperlipidemia refers to lipid metabolism disorders, abnormal fat metabolism or transport, too high lipid concentration in plasma, including total cholesterol and triglyceride levels, higher than those of normal people of the same age, the said composition comprising:
i. akebia fruit in an amount of 5-10 parts by weight,
ii. curcuma root in an amount of 6-12 parts by weight,
iii. pomelo in an amount of 6-12 parts by weight,
iv. fructus aurantii in an amount of 6-12 parts by weight,
v. fructus aurantii immaturus in an amount of 6-12 parts by weight,
vi. star anise in an amount of 6-12 parts by weight,
vii. murraya paniculata in an amount of 6-12 parts by weight,
viii. aspongopus in an amount of 6-12 parts by weight,
ix. sword beans in an amount of 6-12 parts by weight,
x. caesalpinia decapetala in an amount of 6-12 parts by weight,
xi. China fir in an amount of 6-12 parts by weight,
xii. finger citron in an amount of 6-12 parts by weight,
xiii. rose in an amount of 5-10 parts by weight,
xiv. green tangerine peel in an amount of 6-12 parts by weight,
sundew in an amount of 5-10 parts by weight,
xvi. trifoliate orange in an amount of 5-10 parts by weight,
xvii. kaki calyx in an amount of 6-12 parts by weight,
xviii. litchi in an amount of 6-12 parts by weight,
xix. litchi nucleus in an amount of 6-12 parts by weight,
xx. magnolia in an amount of 6-12 parts by weight,
xxi. cyperus rotundus in an amount of 6-12 parts by weight,
xxii. citron fruit in an amount of 2-4 parts by weight,
xxiii. Chinese buckeye seed in an amount of 6-12 parts by weight,
xxiv. salvia multiorrhiza in an amount of 2-4 parts by weight,
xxv. polygonum orientale in an amount of 6-12 parts by weight,
xxvi. motherowrt fruit in an amount of 3-6 parts by weight,
xxvii. euonymus alatus in an amount of 2-4 parts by weight,
xxviii. campsis grandiflora in an amount of 6-12 parts by weight,
xxix. rhizome of Sichuan lovage in an amount of 6-12 parts by weight,
xxx. medical cyathula root in an amount of 6-12 parts by weight,
xxxi. vaccaria in an amount of 6-12 parts by weight,
xxxii. Chinese rose flower in an amount of 6-12 parts by weight,
xxxiii. Balsamine in an amount of 5-10 parts by weight,
xxxiv. Safflower in an amount of 6-12 parts by weight,
xxxv. Azaleas in an amount of 5-10 parts by weight,
xxxvi. semen brassicae campestris in an amount of 1-2 parts by weight,
xxxvii. caesalpinia sappan in an amount of 6-12 parts by weight,
xxxviii. gleditsia spine in an amount of 5-10 parts by weight,
xxxix. myrrh in an amount of 6-12 parts by weight,
xl. caulis spatholobi in an amount of 5-10 parts by weight,
xli. frankincense in an amount of 6-12 parts by weight,
xlii. herba lycopi in an amount of 6-12 parts by weight,
xliii. selaginella in an amount of 6-12 parts by weight,
xliv. tanarius major in an amount of 5-10 parts by weight,
xlv. peach seed in an amount of 6-12 parts by weight, and
xlvi. licorice in an amount of 9-18 parts by weight.

2. The composition of claim 1, wherein the composition is in a form of a concentrated oral solution.

3. A method for treating Qi stagnancy and blood stasis type of hyperlipidemia, the method comprising administrating an effective amount of a medicinal composition to a subject in need thereof, wherein said medicinal composition is prepared by a method comprising,
i. weighing predetermined amount of each of akebia fruit, curcuma root, pomelo, fructus aurantii, fructus aurantii immaturus, star anise, murraya paniculata, aspongopus, sword beans, caesalpinia decapetala, China fir, finger citron, rose, green tangerine peel, sundew, trifoliate orange, kaki calyx, litchi, litchi nucleus, magnolia, cyperus rotundus, citron fruit, Chinese buckeye seed, Salvia multiorrhiza, polygonum orientale, motherwort fruit, euonymus alatus, campsis grandiflora, rhizome of Sichuan lovage, medical cyathula root, vaccaria, Chinese rose flower, balsamine, safflower, azaleas, semen brassicae campestris, caesalpinia sappan, gleditsia spine, myrrh, caulis spatholobi, frankincense, herba lycopi, selaginella, and tanarius major, peach seed and licorice;
ii. blending the provided raw materials into 1800 ml of water, and soak for 30 minutes;
iii. boiling the medicinal herbs with gentle heat for 30 minutes;
iv. filtering the residue to achieve 390 ml of liquid.
